(19) United States
(12) Patent Application Publication
(10) Pub. No.: US 2018/0110579 A1
(43) Pub. Date: Apr. 26, 2018

(54) INTEGRATED TRAY AND WRAP SYSTEM AND METHOD OF MAKING

(71) Applicant: Allegiance Corporation, Wauekgan, IL (US)

(72) Inventors: VARSHA KALYANKAR, KENOSHA, WI (US); ANDREW M ROLSMA, Libertyville, IL (US); ELIEZER APONTE, El Paso, TX (US); NICHOLAS GUS FOTIS, McHenry, IL (US); DANNY W PENNY, Third Lake, IL (US)

(73) Assignee: Allegiance Corporation, Waukegan, IL (US)

(21) Appl. No.: 15/793,034

(22) Filed: Oct. 25, 2017

Related U.S. Application Data
(60) Provisional application No. 62/412,808, filed on Oct. 25, 2016.

(51) Int. Cl. A61B 50/33; B65D 81/38; B65D 65/12; A61M 5/00

(52) U.S. Cl. CPC A61B 50/33; B65D 81/3813; B65D 65/44; A61M 5/002; B65D 65/12

(57) ABSTRACT

An integrated tray and wrap system for supplying components used in medical procedures is provided and a method of manufacturing the tray while filling and sealing in one continuous process. The trays can be formed on a process line, such as by a thermo-form-fill-seal technique, and once the cavity of the tray is formed, the desired medical components can be loaded into the tray cavity as the formed trays are advanced down the process line. After the components are loaded, a pre-folded drape or non-woven sheet can be affixed to an inner ledge of the tray along an inner fenestration of the drape or sheet, thus attaching the drape or sheet to the tray. Once the drape has been attached, the tray continues to advance along the process line to receive a cover film that can be heat-sealed to the outer rim of the tray.

### FIELD

An integrated tray and wrap system used for medical kits and the method of making said tray system is provided herein. A process for forming the trays, filling, and attaching the wrap or drape in-line and in one continuous process and at one location is provided herein.

### BACKGROUND

In the medical field, doctors and medical personnel often use medical supply packages or kits that have been prepared ahead of time and contain the tools and instruments needed for a certain procedure to be conducted by the doctor and/or medical personnel. These kits or trays are often pre-packaged with the tools and instruments needed for the surgical procedure and then sealed and sterilized. These kits are single use items and after the medical procedure is finished these trays are discarded. In addition, a drape or sheet is often provided with the tray to provide for a sterile barrier once the tray has been opened. The procedure of using a drape to cover a patient and/or the surrounding area provides a sterile field during the surgical procedure. In particular, the drape can be opened up and used to cover an instrument table that will receive the pre-packaged tray/kit and to further provide a sterile surface for placing the tools and instruments thereon.

These pre-packaged trays or kit are often assembled by hand after the tray has been formed. Typical methods of forming the tray can include thermoforming processes or injection molding of the tray. Once the tray has been made, then it can be removed from the machine line and transferred to another location to be filled by hand, e.g., by a person or operator, with the appropriate instruments and drape added. These steps of making the tray, filling the tray and then closing/sealing the tray can happen independently of one another, making for segregated or partitioned process steps. After the pre-packaged tray or kit has been fully assembled and sealed, the sealed tray or kit can then be packaged in a shipping carton or container for transportation to its final location.

Typically the drape is folded while on the line, e.g., the operator must fold the drape to a smaller dimension while on the line filling the tray with the instruments. The drape is typically wrapped around the tray, enclosing the tray opening and acting as a cover to the tray until it is unwrapped and opened. Alternatively, the drape can also be folded into a smaller footprint that fits inside the opening of the tray, where the folding of the drape takes place on-line by the operator, and a cover or film seal can be placed over the opening of the tray to close it. Once the tray has been filled and sealed or closed, then it can be sterilized, if necessary for the medical procedure.

Due to the steps of manufacturing the tray first and then sending it off to a separate location for filling and sealing, there are many extra steps that are incurred not to mention the length of time it takes to complete a pre-packaged tray or kit can be excessively long.

### SUMMARY

A pre-packaged medical tray is provided with two sealing flanges or ledges along the perimeter of the tray; an inner flange for sealing a drape thereto and an outer flange for sealing a cover film thereto. The pre-packaged medical tray can be filled with the necessary medical supplies and instruments and then covered with a folded drape or wrap, which is sealed to the inner flange. The inclusion of all of the needed supplies, e.g., instruments and drape, in the interior of the tray and then sealed, provide for reduced steps for a clinician to access the components of the tray, reduces storage space due to a compact size and the drape being included in the tray package, and maintain sterility of the drape and all instruments when sterilized, among other benefits.

The drape that is included in the interior of the tray cavity can have a fenestration that is approximately the size of the opening of the tray cavity and will enable the user to have direct access to the interior of the tray and in turn the components stored in the cavity of the tray. It is at the edges of the fenestration that the drape can be attached to the inner ledge of the tray using any conventional attachment or adhesive method. The fenestration can be located anywhere on the drape and, in one aspect, can be at a central or middle location. The drape also can be pre-folded prior to receiving the drape on the process line for insertion into the tray cavity, thus, eliminating a step of having an operator fold the drape at the time it is being inserted into the tray. The drape can be folded to a size or dimension that is similar to the dimension of the tray cavity such that the drape can fit inside of the tray cavity. When the drape is attached to the inner ledge of the tray along the edges of its fenestration, it will be positioned inside of the tray such that the drape rests on the inner ledge of the tray rather than being contained inside of the tray compartment along with the components or instruments packaged in the tray.

The tray can be manufactured, filled and sealed in a continuous in-line process, such as a process performed on a thermo-form-fill-seal machine (TFFS). The tray can be thermoformed in-line with all subsequent process steps performed downstream and in-line on the process lines. The thermoformed trays can remain attached as they move down the line in a machine direction to be filled with the necessary components. The trays are formed to have a sufficient depth to accommodate all of the supplies, instruments, etc. that are to be stored therein. As the trays are formed, they are formed with a stepped rim, e.g., having two flanges around the perimeter. An outer flange is positioned toward the top or upper portion of the tray, about its perimeter, and an inner flange is positioned slightly inward of the top or outer flange, e.g., toward the base of the tray. The space or area between the outer and inner flange is such that the space between the two flanges can completely accommodate the folded drape.

After the trays are filled with the necessary components, the pre-folded drape can be affixed or attached to the inner ledge of the tray by any attachment method that is appropriate, such as hot melt or another adhesive or another method. The drape is attached to the inner ledge along the fenestration of the drape, e.g., the edges of the fenestration are attached to the inner ledge. Thus, when the drape is unfolded upon opening, the fenestration lines up with the cavity of the tray to allow access to the components stored therein.

After the drape is attached, then a film or lid can be sealed to the upper flange of the tray using any appropriate attachment method. In one aspect, the lid material is a heat-sealable film that seals to the outer flange of the tray when heat is applied to the area of the lid film that is in contact with the outer flange, effectively sealing it to the tray at the outer flange area. The lid is sealed to the tray flange to help maintain sterility of the components of the tray during transportation and until the tray is opened by the user. After sealing the tray closed, the trays and lid film are separated. The sealed tray packages can then be sterilized if desired. Moreover, once the sealed trays or kits are separated from one another they can be packaged in an outer package for shipping purposes, such as placement in a shipping carton or container for transportation to another location.

Forming the trays in-line and also during the same process filling the trays with the desired components and a drape, allows the drapes to be loaded at the same time the trays are being made. This provides for a faster method of making and filling pre-packaged medical tray packages and an easier process that has less steps involved, thus reducing the transferring of trays to different lines for filling and sealing. Moreover, having the pre-folded drape contained inside of the tray avoids the need to provide an outer bag or plastic wrap to protect a drape that previously may have been wrapped around the outer surface of the tray. The drape and components all contained inside the tray can provide for substantially instantaneous set-up of the tray, drape and components in a manner that maintains sterility of the tray contents and field. Additionally, the tray fold pattern is such that an easy open can be effected with minimal handling of the drape as well as providing a direct access to the contents of the cavity of the tray without having to detach the drape from the tray.

### BRIEF DESCRIPTION OF THE DRAWINGS

FIG. 1 is a perspective view of a tray as disclosed herein;

FIG. 2 is a cross-sectional view of the tray of FIG. 1 taken along line 2-2;

FIG. 3 is a cross-sectional view of a filled and sealed tray;

FIG. 4 is a perspective view of a filled tray after it has been opened and the pre-folded drape unfolded;

FIG. 5 is a perspective view of a process line for making, filling and sealing the tray of FIG. 1;

FIG. 6 is a cross-sectional view of the process line shown in FIG. 5;

FIG. 7 is a process flow diagram of a process for making, filling and sealing the tray of FIG. 1; and

FIGS. 8A-8D are a cross-sectional view of an alternate embodiment of a forming station used on the process line.

### DETAILED DESCRIPTION

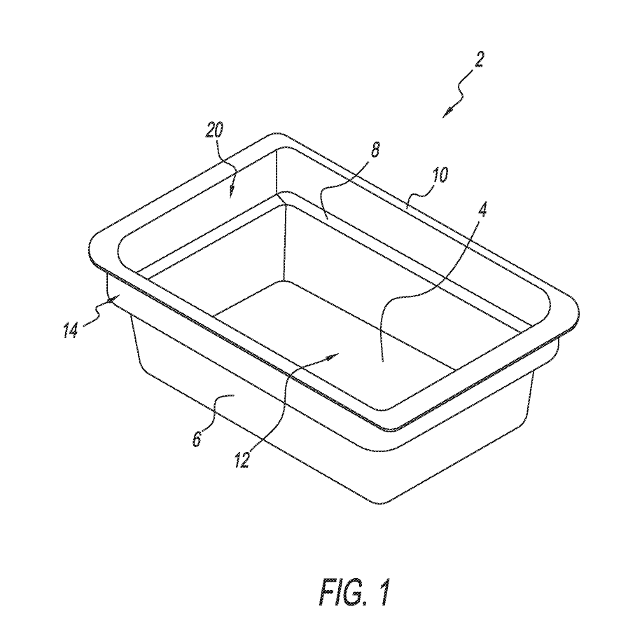

An integrated tray and drape system for the pre-packaging of medical supplies and/or instruments and a method of making these medical supply trays are described herein and provided in FIGS. 1-8D. A tray is thermoformed with two sealing flanges, an inner and an outer flange. The inner flange allows the drape to be sealed to it as well as providing a resting ledge to hold the drape and suspend it thereon, preventing the drape from falling into the inner cavity of the tray which contains the medical supplies. A process is further provided for making, filling and sealing the medical supply trays in one, continuous in-line process.

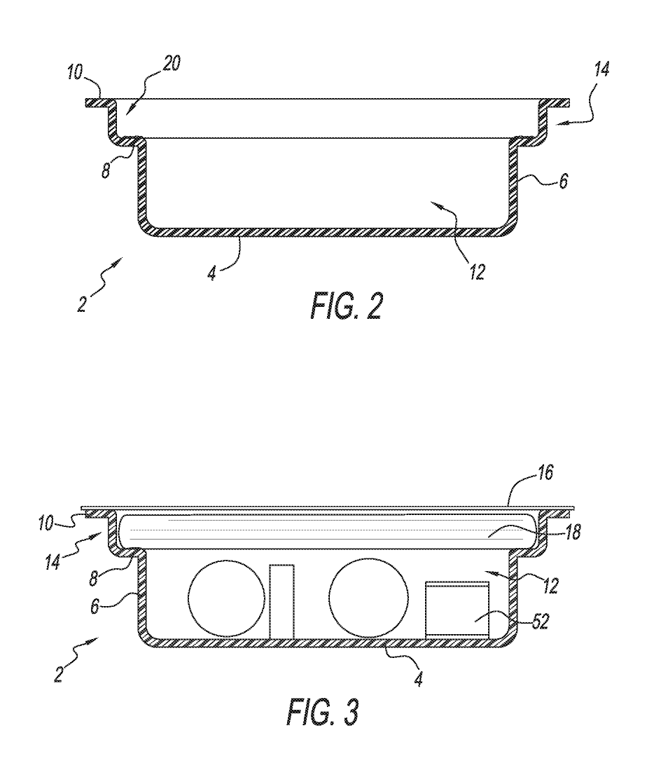

Turning to FIGS. 1 and 2, the tray 2 comprises a base 4 with upstanding sidewalls 6 therefrom. Along the outer periphery of the tray, at an upper end 14 of the sidewalls 6, there is an outer rim or ledge 10 that defines the opening 20 of the tray. The sidewalls 6 can further be provided with a stepped inner rim or ledge 8 that extends peripherally around the base of the tray along the inner edge of the sidewalls 6 and is spaced from the outer ledge 10. The inner ledge 8 is positioned on the inner sidewalls 6 at a distance that is closer to the opening 20 than the base 4. The sidewalls 6 between the base 4 and the inner ledge 8 define an interior section or cavity 12 for receiving items therein. The sidewalls 6 between the inner ledge 8 and outer ledge 10 define a space for receiving and storing a folded drape. In one aspect, the opening 20 is larger in perimeter than the perimeter of the cavity 12.

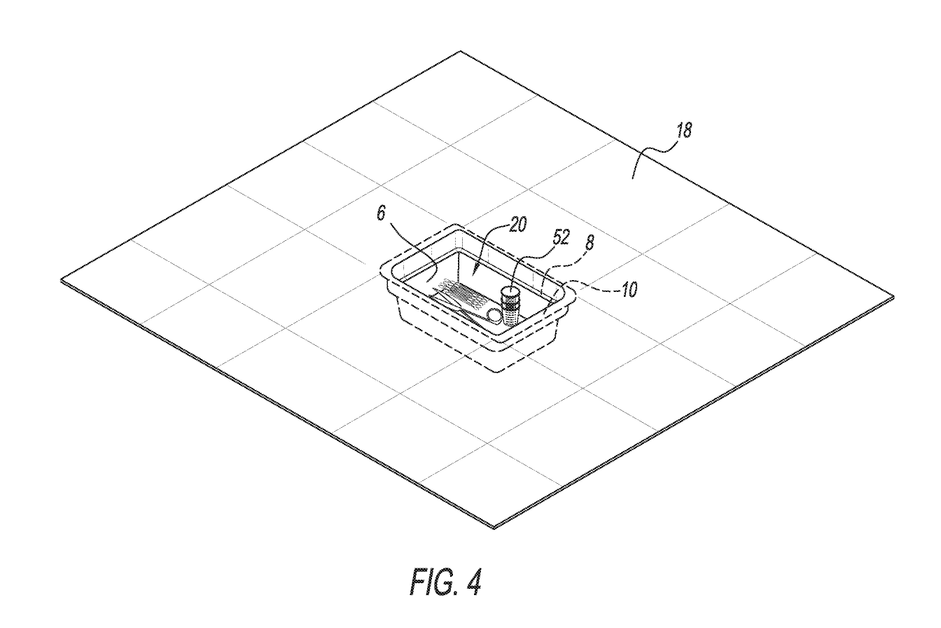

Turning to the cross-sectional view shown in FIG. 3, the tray 2 is shown sealed with a cover film 16, and the interior cavity 12 can be filled with several items 52 used in a medical procedure, which are nestled along the base 4 of the tray and contained in the tray cavity 12. To open the tray 2 and access the items stored in the cavity 12, one must simply lift up and tear off the lid film, unpeeling it from the outer flange 10 to which it has been sealed and then unfold the drape to expose the fenestration and the interior of the tray. In one aspect, one section of the cover lid can be unsealed, such that it can be used as a gripping tab for the user to grasp as they lift off the film. In another aspect, one corner can be left unsealed or in yet another aspect a portion or edge of the outer flange can be left unsealed.

The tray 2 can be used to store various medical supplies, instruments, tools and the like that may be useful in a medical procedure. Some non-exhaustive examples can include gloves, face masks, bulb syringes, forceps, scissors, tourniquet, medical instruments, gauze, iodine wipes and bottles, topical skin prep applicators, tape measure, pen, bandages and dressings, and other similar components.

The tray 2 can be shaped and sized appropriately such that the cavity of the tray will house the desired components of the tray. In one aspect, the tray can be rectangular. In another aspect, the tray could be square or any other shape that best fits the tray components that are to be later added. The tray can be any size that is appropriate for the use. The tray can have dimensions that range from about 3 inches to about 50 inches in length, from about 3 inches to about 50 inches in width, and about 0.5 inches to about 10 inches in depth. In one aspect, the tray can have a dimension of about 7.5 inches length×about 5 inches width×about 3 inches depth and is rectangular in shape. In another aspect, the tray can have a dimension of about 10 inches length×about 7.5 inches width×about 5 inches depth. The stepped ledge of the tray, e.g., the outer flange 10 and the inner flange 8 can be provided such that the outer flange 10 is spaced a distance of about 0.5 inches to about 1 inch from the inner flange 8 or an appropriate distance such that the pre-folded drape can fit within the space between the two flanges, such that the spaced distance between the flanges is similar to the depth of the folded wrap. In one aspect, the inner flange 8 is spaced from the outer flange 10 at a distance of about 0.75 inches. In yet another aspect, the inner flange 8 and outer flange 10 can be spaced about 1 inch.

The drape 18 can be pre-folded prior to bringing it in-line and prior to inserting it into the tray 2. This avoids having an operator manually fold the drape in-line either around the tray package or to a smaller dimension to be inserted into the tray. The drape 18 can be folded in any manner that provides the drape 18 in a reduced size that will fit into the opening 20 of the tray 2, yet large enough such that it rests on and is supported on the inner ledge 8 of the tray 2, without extending significantly beyond the inner ledge 8 and into the cavity. In another aspect, the drape 18 can be provided in a folded pattern that is smaller than the inner ledge 8 of the tray, such that it would rest at least partially in the cavity 12 of the tray. In yet another aspect, the drape 18 can be provided in a fan fold comprising of alternating folds along the length of the drape, and opposite fan folds along the width of the drape. The drape 18 can be fan folded and/or book folded, where book folded is folding over a drape along a center line onto another portion of the drape such that it is oriented similarly as the cover of a book, so that the folded drape fits on the inner ledge allowing for attachment, without falling into the inner cavity. In another aspect, it can be folded by using a fixture. In yet another aspect, it can be folded automatically using a machine. Folding can occur remotely in another location or at the same location as the forming of the trays or even as an added step or station on the process line. The folded drape 18 can have a thickness that is comparable to the space between the inner flange 8 and the outer flange 10. In one aspect, the folded drape 18 can have a thickness of about ⅛ inches to about 1.5 inches. In another aspect, the folded drape 18 can have a thickness of about 0.5 inches. In yet another aspect, the pre-folded drape can comprise a drape that is partially folded, such that it still may require further folding upon placement in the tray. Pre-folded can refer to a drape that is folded any number of times but at least once such that it may not be completely folded into its final configuration for placement in the tray. Alternatively, a pre-folded drape may be a drape that is folded completely into its final configuration for placement in the tray.

The drape 18 can also have a fenestration 22 located within an interior portion of the drape 18. In one aspect, the fenestration 22 can be in a central or middle area of the drape 18. The size of the fenestration 22 can generally match the size of the perimeter of the inner ledge, such that the drape can be attached to the inner ledge 8 without excess drape material hanging off of the ledge 8. The folding of the drape 18 is provided in a manner such that the fenestration 22 of the drape is exposed along a central lower portion of the folded drape, such that upon placement of the drape in the tray the edges of the fenestration 22 are exposed and can be easily attached to the inner flange 8 of the tray. The material of construction of the drape 18 can be any material that is appropriate for use in a medical facility and during a medical procedure, such as nonwoven SMS fabric (spunbond-meltblown-spunbond). In one aspect, the drape 18 can be a non-woven sheet that is made of between about 18 to about 35 gsm (grams per square meter) spunbond-meltblown-spunbond nonwoven fabric, where the spunbond and meltblown composition can be polypropylene. In another aspect, the drape 18 can be a non-woven sheet that is about 25 gsm. The non-woven sheet can be further treated to be electro-static, such as with an anti-stat treatment. In another aspect, the drape 18 can comprise a paper and/or poly laminate material, or a Tyvek material, or a white wet laid nonwoven fabric, such as composed of natural cellulose and/or synthetic blended fibers. Once the drape 18 has been pre-folded, it can be attached to the inner ledge 8 of the tray via any appropriate attachment method. In one aspect, a hot melt adhesive can be applied to the upper surface of the inner ledge 8 and the drape 18 can be attached thereto along the fenestration of the drape at an undersurface of the fenestration. In another aspect, a machine or robot can apply an adhesive to the inner ledge 8. In yet another aspect, the adhesive can be applied manually. In an alternative method, the folded drape can be attached to a plastic injection molded frame that is then attached to the tray.

After all of the components have been inserted into the cavity of the tray and the fenestrated and folded drape 18 has been inserted over the top of the inserted components, such that the fenestrated and folded drape 18 covers the components, a cover film or lid can be attached to the upper ledge 10 of the tray to seal and close the tray and its components. The closed tray package can also be sterilized if desired. Conventional sterilization methods can be used, such as ethylene oxide (EO) sterilization, gamma radiation, ebeam radiation, STERRAD®, dry or steam heat.

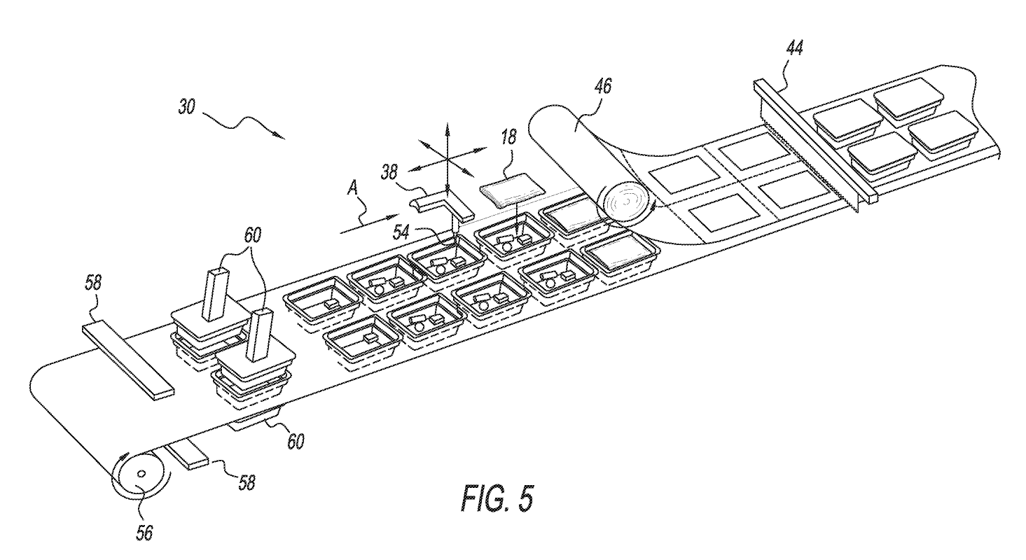

When the tray is ready for use, the user simply peels off the lidding material or cover film 16 and unfolds the pre-folded drape 18, as shown in FIG. 4. This provides access to the interior cavity of the tray through the fenestration of the drape 18.

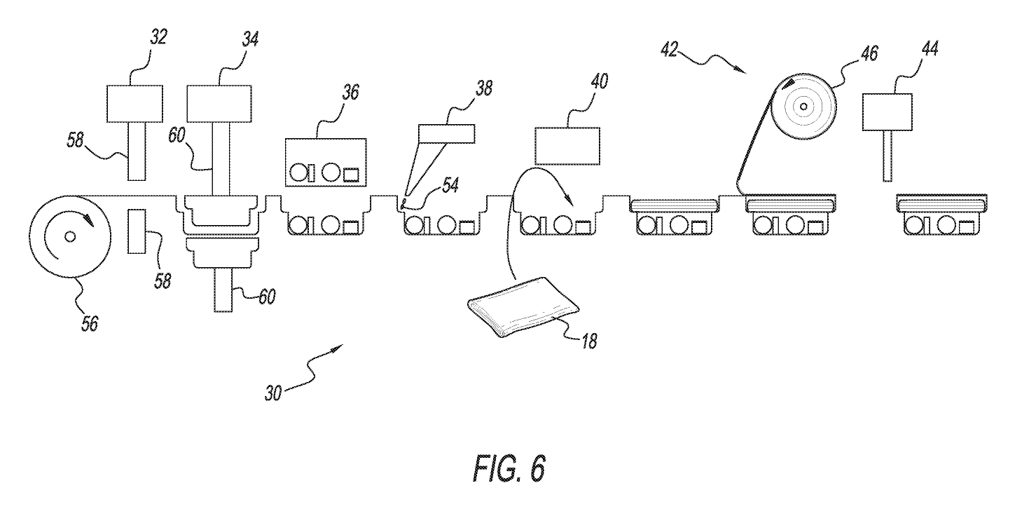

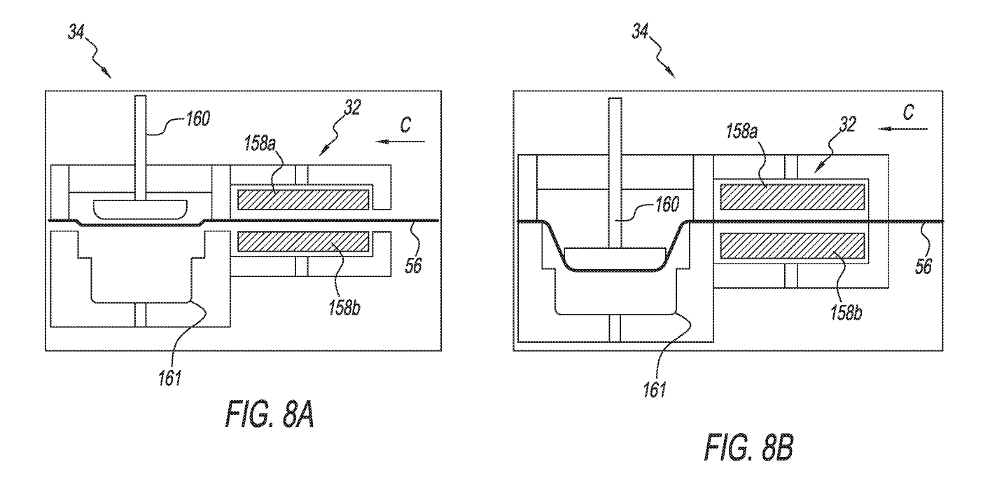

A method of making, filling, and sealing the medical supply trays is provided in FIGS. 5-7. Turning to FIG. 7, a flow diagram is shown of the general process steps involved with making the package. At Step 1, the trays having a cavity and a stepped ledge, e.g., inner and outer ledge, are formed on a packaging machine. Once the trays are formed, the desired components are inserted into the tray cavity at Step 2. At Step 3, an adhesive can be applied to the inner ledge of the tray. At Step 4, the pre-folded drape 18 is attached to the adhesive on the inner ledge. Alternatively, instead of the adhesive being applied at Step 3 to the inner ledge of the tray, it can instead be placed upon the drape 18 such that the inner ledge of the tray is attached to the drape via the adhesive on the drape. Still alternatively, another type of fastener or attachment means can be employed at Step 3 that does not require adhesive or is used in combination with adhesive. Some examples of alternate attachment means can include a rigid piece that holds the drape in place by friction fit or snapping into the tray, a drape that can be heat sealed to the tray to the inner ledge 8, or any other appropriate attachment means. At Step 4, this alternative attachment means can affix the drape to the tray. After the tray is filled with all of the desired items, then the top web film is applied and sealed to the outer ledge of the tray to close the tray at Step 5. At Step 6, the attached trays are separated by any appropriate cutting or singulation method.

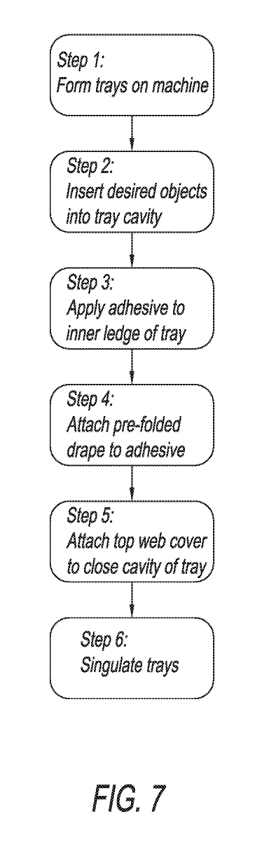

In FIGS. 5-6, an overview of the process is shown where a thermoform-fill-seal machine 30 (TFFS) is depicted. A thermoform film roll or sheet 56 can be provided and feeds a continuous length of film or a continuous length of sheet in a machine direction A toward a pre-heat station 32. At the pre-heat station 32, the film or sheet roll 56 can be heated to its softening point by passing under pre-heat plates 58, using radiant heat, for example, to make it malleable. The softened film or sheet roll material can then allow for being formed into the required shape at the forming station, which can form the tray by any known process, such as, for example by vacuum, air, or plug assist. The thermoform roll 56 can comprise any appropriate material for forming a rigid or semi-rigid tray, such as a polymer or plastic material typically used in the formation of medical trays, or a material which may also comprise a recycled component of a polymer or plastic material typically used in formation of medical trays. In one instance, the film or sheet roll material can be made by any known process, such as, for example, extrusion or co-extrusion, and the film or sheet roll material may comprise a material or combination of materials that can be selected from the group consisting of polypropylene, polyvinyl chloride (PVC), amorphous polyethylene terephthalate (APET), high-density polyethylene (HDPE), high impact polystyrene (HIPS), polyethylene terephthalate glycol (PETG), foamed polystyrene, including other materials such as other polyolefin materials, and the like.

As the roll of film or sheet 56 passes through the pre-heat station 32 the pre-heat plates 58 are heated up to provide an initial warming of the film or sheet 56 before it enters the forming station 34. The pre-heat plates 58 can be provided at any adequate temperature to provide the desired pre-heat of the film, without being too hot that the film becomes excessively malleable or too soft. In one aspect, the temperature of the pre-heat plates 58 can vary between about 185° F. and about 500° F., and in a preferred aspect, the temperature of the pre-heat plates 58 can be at about 210° F. to about 290° F.

At the forming station 34, the pre-heated film advances in machine direction A toward the forming dies 60 where the pre-heated film can be formed into the desired shape by pressing it with a forming die having the desired shape. The forming dies 60 can apply heat to the film and can apply a force to the film to shape the film and form a cavity therein having a shape similar to the forming dies 60, resulting in a web of trays attached to one another along the outer flanges. In another aspect, the forming die can press against the pre-heated film and can further apply an air vacuum to aid in forming the desired shape in the film. In yet another aspect, the forming station 34 can include forming methods comprising one or more of air, mechanical or vacuum methods all being applied at the same or substantially the same time to form the tray.

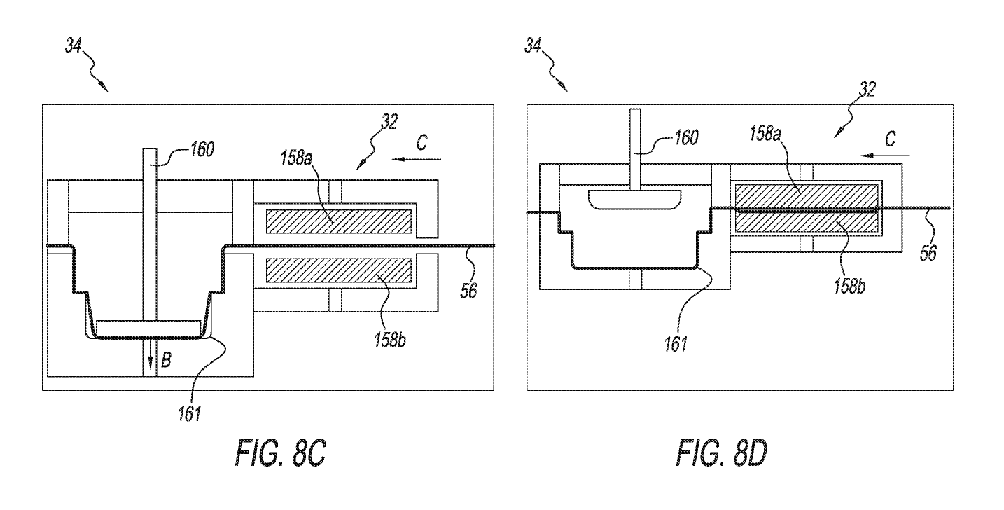

In one example, as shown in FIGS. 8A-8D, a pair of plugs or dies can apply a force upon the tray, where an upper die 160 can have a general plug shape and a lower die 161 can have a shape that is closer in shape to the final tray design, such that the tray is shaped more by the lower die rather than the top die. At the same time as the dies are applying a force to the film or sheet, an air pressure and/or a vacuum can also be applied such that the force of the air pressure and/or vacuum helps to further shape the tray to the desired final shape. FIG. 8A shows a film being passed between preheat dies 158a and 158b without touching the preheat dies as it enters the forming station 34. Alternatively, the preheat dies can comprise a single plate along the bottom or underside of the film. In FIG. 8B, the upper die 160 begins to advance in a downward direction to contact the film 56, at this same time the film entering the preheat station 32 is positioned closer to the preheat dies. Turning to FIG. 8C, the upper die or plug 160 has pushed the film 56 as far down as the plug 160 can extend, however, application of vacuum, as represented by arrow B, is applied at this point to pull the film 56 down to extend into the bottom die 161. In FIG. 8D, as the film has been fully formed to the tray shape and the plug 160 is retracted, the vacuum pressure is stopped; at this final step the film is in contact or almost in contact with the preheat dies 158a and 158b. Upon cessation of the vacuum pressure, an air flow can be blown upward to help dislodge the tray from the bottom die. A similar forming process can also be used with a sheet instead of a film.

After the attached trays are formed, they advance to the loading station 36, where the tray components 52 are loaded. These components 52 can be loaded by an operator or loaded by a robot or other similar automated method. As mentioned above, the components 52 that are loaded into the tray cavity can comprise any appropriate tool, instrument, or supply that can be used in a medical procedure.

After the medical components 52 are loaded into the cavity of the tray, the loaded tray advances in machine direction A toward the adhesive application station 38. At the adhesive application station 38, a glue or other adhesive can be applied to the inner flange 8 of the loaded tray in preparation for receiving the pre-folded drape. The adhesive can be applied by any appropriate method. In one aspect, the adhesive can be applied manually. In another aspect, the adhesive can be applied by a machine attachment to the TFFS machine that dispenses adhesive. In yet another aspect, the adhesive can be applied by a robot, such as by a robotic arm, that can deliver controlled application of the adhesive such that it is only placed along the perimeter of the inner flange 8. In still yet another aspect, the adhesive can be applied by an XYZ system that has nozzles moving along rails and the nozzle can dispense the adhesive as it moves in an x, y, z direction. The adhesive can comprise any appropriate adhesive used for medical trays and that can withstand possible sterilization techniques. For example, the adhesive can comprise a material such as hot-melt adhesive, glue, thermoplastic glue, pressure sensitive adhesive, tackifiers, and the like.

After the adhesive has been applied, the tray can continue to advance in a machine direction to the drape placement station 40. The pre-folded drape 18, as already described herein, is placed into the opening 20 of the tray and attached to the inner flange 8 via the previously applied adhesive. The drape 18 is attached to the tray along the edges of the drape fenestration 22. The drape 18 can be attached to the tray in any appropriate manner, such as by an operator, by a robot, or by any other automated method that is appropriate. In an alternative embodiment, the adhesive can instead be applied to the drape such that the tray is attached to the drape along the edges of the drape fenestration 22. In yet another alternative embodiment, the drape 18 can be affixed to the tray without using adhesive. For example, the drape can be affixed to the tray by an injection molded part which snaps into the tray and attaches or affixes the drape to the tray. In yet another example, the drape can be affixed to the tray via heat sealing methods.

Once the drape 18 has been attached to the tray, the tray can continue to advance to the sealing station 42. A roll 46 of cover film or lidding material is provided and fed over the filled trays and the film and tray are fed past sealing dies 62 that contact the cover film at the outer flange area of the tray to heat up the edges of the cover film that overlay the outer flange, thus providing a seal between the cover film and the outer flange of the tray. The heat sealing dies 62 are sized such that the dimension and orientation of the die matches the dimensions of the outer flange of the tray, thus, being able to line up with the outer flange when heat-sealing the cover film thereto. The cover film can comprise any appropriate material for sealing a medical supply package and for withstanding conventional sterilization methods. The cover material preferably is a material that is itself heat-sealable. In one aspect, the cover material can comprise high-density polyethylene fibers, such as Tyvek®, or paper, plastic, or other similar materials. In one aspect, the cover film or lidding material can be provided as separate, pre-cut lidding material rather than a roll.

Upon sealing the trays, they can then be advanced to the cutting station 44. At the cutting station 44 the attached trays are singulated by a cutting blade or other similar device that separates and cuts the packages apart resulting in the individually sealed and packed trays. After the trays have been sealed and separated, the trays can be sterilized if necessary for the type of medical procedures the trays will be used for and can also be placed in an outer package for shipping to another location. Any conventional sterilization methods can be applied. For example, some methods may comprise ethylene oxide (EO), gamma, ebeam, STERRAD®, or steam sterilization. In particular, EO sterilization can be utilized.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification be considered as illustrative only, with a true scope and spirit being indicated by the claims and their full scope of equivalents.

What is claimed is:

1. A method of forming, filling and sealing an integrated tray and wrap system, the steps comprising:
feeding a web from a roll of film or sheet in a machine direction toward a pre-heat station;
heating the web to pre-heat the film or sheet as it advances toward a forming station;
forming the web of film or sheet into a series of attached trays having a cavity and a stepped rim comprising an outer flange positioned at an opening of the tray and an inner flange positioned at an interior of the tray;
loading tray components into the interior of the tray as the tray continues to advance in the machine direction to a wrap insertion station;
affixing a pre-folded wrap to the tray such that the pre-folded wrap is contained in a space between the inner flange and the outer flange;
feeding a cover film over the opening of the tray and advancing toward a sealing station;
sealing the cover film to the outer flange of the tray; and
singulating the attached trays into individually sealed and filled trays.

2. The method of claim 1, wherein the step of affixing a pre-folded wrap comprises applying an adhesive to the inner flange of the tray and attaching a pre-folded wrap to the inner flange of the tray along the adhesive.

3. The method of claim 2, wherein the step of applying adhesive is selected from the group consisting of manual application, a machine attachment, and a robotic arm.

4. The method of claim 2, wherein the adhesive can be applied along portions of a wall of the tray between the outer and inner flanges.

5. The method of claim 1, wherein the step of attaching the pre-folded wrap comprises attaching the pre-folded wrap to the inner flange at an outer edge of a fenestration located within an interior area of the pre-folded wrap.

6. The method of claim 1, wherein the pre-folded wrap is folded in either a fan fold, a book fold pattern or both.

7. The method of claim 1, wherein the material of construction of the wrap is a non-woven spunbound-meltblown-spunbound fabric.

8. The method of claim 1, wherein the space between the inner flange and the outer flange comprises a distance of about 0.5 inches to about 1 inch.

9. The method of claim 1, wherein the space between the inner flange and the outer flange is a distance equal to a thickness of the pre-folded wrap.

10. The method of claim 1, wherein the web of film or sheet has a material of construction selected from one or more materials selected from the group consisting of polypropylene, polyvinyl chloride (PVC), amorphous Polyethylene (APET), high-density polyethylene (HDPE), high impact polystyrene (HIPS), polyethylene terephthalate glycol (PETG), and foamed polystyrene, and other polyolefin materials.

11. The method of claim 1, wherein the cover film has a material of construction selected from the group consisting of high-density polyethylene fibers, such as Tyvek®, or paper, plastic.

12. The method of claim 1, wherein the tray components comprise one or more items selected from the group consisting of gloves, face masks, bulb syringes, forceps, scissors, tourniquet, medical instruments, gauze, iodine wipes and bottles, topical skin prep applicators, tape measure, pen, bandages and dressings.

13. A packaging machine for forming, filling and sealing an integrated tray and wrap system, the packaging machine comprising:
a forming station for forming a web of trays having a cavity and a stepped rim comprising an outer flange positioned at an opening of the tray and an inner flange positioned at an interior of the tray;
a component insertion station for loading medical components into the cavity of the tray;
an adhesive station for applying adhesive to at least a portion of the inner flange of the tray;
a wrap insertion station for attaching a pre-folded wrap to the adhesive-containing inner flange;
a top-web sealing station for attaching a cover lid to the outer flange and sealing the tray; and
a cutting station for separating the sealed cover lid and web of trays into individually-sealed trays.

14. The packaging machine of claim 13, wherein the adhesive station further comprises the step of applying adhesive selected from the group consisting of manual application, a machine attachment, and a robotic arm.

15. The packaging machine of claim 13, wherein the adhesive is a material selected from the group consisting of hot-melt adhesive, glue, thermoplastic glue, pressure sensitive adhesive, and tackifiers.

16. The packaging machine of claim 13, wherein the wrap insertion station further comprises the step of attaching the pre-folded wrap to the adhesive-containing inner flange by attaching an interior edge of a fenestration of the pre-folded wrap to the inner flange.

17. A method of forming, filling and sealing an integrated tray and wrap system, the steps comprising:
feeding a web from a roll of film or sheet in a machine direction toward a pre-heat station;
heating the web to pre-heat the film or sheet as it advances toward a forming station;
forming the web of film or sheet into a series of attached trays having a cavity and a stepped rim comprising an outer flange positioned at an opening of the tray and an inner flange positioned at an interior of the tray;
loading tray components into the interior of the tray as the tray continues to advance in the machine direction;
applying an adhesive to the inner flange of the tray and advancing the tray to a wrap insertion station;
attaching a pre-folded wrap to the inner flange of the tray along the adhesive such that the pre-folded wrap is contained in a space between the inner flange and the outer flange;
feeding a cover film over the opening of the tray and advancing toward a sealing station;
sealing the cover film to the outer flange of the tray; and
singulating the attached trays into individually sealed and filled trays.

18. The method of claim 17, wherein the adhesive can be applied along portions of a wall of the tray between the outer and inner flanges.

19. The method of claim 17, wherein the step of attaching the pre-folded wrap comprises attaching the pre-folded wrap to the inner flange at an outer edge of a fenestration located within an interior area of the pre-folded wrap.

20. The method of claim 17, wherein the space between the inner flange and the outer flange is a distance equal to a thickness of the pre-folded wrap.
